(19) United States
(12) Patent Application Publication
(10) Pub. No.: US 2018/0184979 A1
(43) Pub. Date: Jul. 5, 2018

(54) ADHESIVE FOR OPTICAL WEARABLE SENSORS

(71) Applicant: The Texas A&M University System, College Station, TX (US)

(72) Inventors: John P. Hanks, College Station, TX (US); Daniel Alge, College Station, TX (US); Akhilesh Gaharwar, College Station, TX (US); Gerard L. Cote, College Station, TX (US); Casey Pirnstall, Fairborn, OH (US)

(73) Assignee: The Texas A&M University System

(21) Appl. No.: 15/398,406

(22) Filed: Jan. 4, 2017

(51) Int. Cl. A61B 5/00; A61B 5/021; A61B 5/024; A61B 5/1455

(52) U.S. Cl. CPC A61B 5/6833; A61B 5/6898; A61B 5/681; A61B 5/6803; A61B 5/6802; A61B 2562/0233; A61B 5/02427; A61B 5/0077; A61B 5/14552; A61B 5/4818; A61B 5/02141

(57) ABSTRACT

An adhesive interface device includes an interface material having a first surface and a second surface, wherein the first surface is configured to adhere to a tissue, the second surface is configured to adhere to a wearable optical sensor device, and the interface material has a refractive index similar to the tissue when illuminated.

### RELATED APPLICATIONS

This application claims priority to U.S. Provisional Patent Application Ser. No. 62/274,647, filed on Jan. 4, 2016, which is specifically incorporated by reference in its entirety herein.

### TECHNICAL FIELD

The present disclosure relates to an adhesive interface device. In particular, it relates to an index matched pressure sensitive adhesive interface device that adheres and optically couples a wearable optical sensor to tissue.

### BACKGROUND

A light emitting diode (LED) and a photodiode are commonly used in combination to measure and continuously log a wide variety of physiological parameters such as heart rate, hydration, blood oxygenation, lactate threshold level, and cuffless blood pressure during physical activity and sleep.

The current LED and photodiode devices use a technical approach commonly implemented in both reflectance and transmissive pulse oximeters to measure changes in absorption of wavelengths of light. A pulse oximeter is a non-invasive continuous measurement device that indirectly monitors the oxygen saturation of a patient's blood and changes in blood volume in the skin and tissue. A typical pulse oximeter utilizes one or more small LEDs to introduce light into the tissue, a photodiode to measure the absorption or reflection of light, and an electronic processor. Pulse oximeter devices can be used on the finger, earlobe, foot, and other bodily locations. Recently, optical devices similar to these have been deployed in wristbands, earbuds, eyeglasses, wristwatches, and smartphones as wearable devices that continuously monitor heart rate and blood oxygenation levels.

### SUMMARY

An embodiment of the disclosure is an adhesive interface device, comprising: an interface material having a first surface and a second surface; wherein the first surface is configured to adhere to a tissue; wherein the second surface is configured to adhere to a wearable optical sensor device; and wherein the interface material has a refractive index similar to the tissue when illuminated. In an embodiment, the first surface and the second surface of the interface material are adhesive. In an embodiment, the interface material is less than 1 mm thick. In an embodiment, the interface material is transparent. In an embodiment, a liner covers the adhesive on the first surface and the second surface of the interface material. In an embodiment, the liner is a waxy release paper. In an embodiment, the tissue is illuminated by a LED. In an embodiment, the refractive index of the interface material is between about 1.3 and 1.5. In an embodiment, the refractive index of the interface material is about 1.3. In an embodiment, the device further comprises a first adhesive on the first surface and a second adhesive on the second surface. In an embodiment, the interface material is a pressure sensitive adhesive. In an embodiment, the pressure sensitive adhesive is at least one selected from the group consisting of synthetic elastomers, hydrogels, polyethylene glycols, acrylics, rubbers, silicones, polyurethanes, polyesters, or polyethers. In an embodiment, the wearable optical sensor device is one of a smartwatch, a smartphone, a fitness band, eyeglasses, a heart rate monitor, an armband, a ring, or ear buds. In an embodiment, the wearable optical sensor device is one of a photoplethysmograph, a cuffless blood pressure monitor, a pulse oximeter, a heart rate monitor, or a device for use related to sleep apnea. In an embodiment, the device has a hole for light transmission for illuminating tissue with one or more wavelengths. In an embodiment, absorption is measured using at least one photodetector. In an embodiment, the interface material is configured to act as a waveguide or diffuser for illuminating tissue with one or more wavelengths. In an embodiment, the material is configured to polarize or filter light for illuminating tissue with one or more wavelengths. In an embodiment, the device is disposable. In an embodiment, the device further comprises a light scattering material to increase light transmission into the tissue.

The foregoing has outlined rather broadly the features of the present disclosure so that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 is a cross-sectional view of an embodiment of the disclosed device illustrating movement of light through an optical adhesive, according to embodiments of the disclosure.

FIG. 2 is a three-dimensional cross-sectional view of the adhesive interface device and light waveguide, according to embodiments of the disclosure.

FIG. 3A-3F are illustrations of various wearable devices with which the adhesive interface device can be used including (A) a watch 301, (B) a fitness wristband 302, (C) a ring 303, (D) a smartphone 304, (E) a finger clamp 305, and (F) an ear buds 306, according to embodiments of the disclosure.

### DETAILED DESCRIPTION

The particulars shown herein are by way of example and for purposes of illustrative discussion of the various embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3rd Edition.

As used herein, the term “a” or “an” can refer to one of or a plurality of the elements it modifies unless it is contextually clear.

Tissue and its various components, such as melanin, oxygenated and deoxygenated blood, and other bodily fluids, absorb light at different wavelengths. Due to this absorption variance, LEDs of wearable pulse oximeter devices are often at various colors or wavelengths (i.e., green, red, and infrared) to measure various tissue parameters and improve measurement quality. For example, a red LED can have a wavelength of 660 nm and an infrared LED can have a wavelength of 940 nm. Absorption of light at these wavelengths can differ significantly between blood saturated with oxygen and blood lacking oxygen; oxygenated hemoglobin absorbs more infrared light, while deoxygenated blood absorbs more red light. A heart beat pulse changes the amount of oxygenated and deoxygenated blood volume in the tissue. The change in absorption can be measured and tracked continuously using a photodiode or photodetector.

The stability of these monitoring systems and their ability to operate in the presence of motion and other noises is a challenge facing users who want to continuously track and log measurements with minimal or no loss of data. During continuous tracking of heart rate and other physiological signals, the LEDs can become decoupled from the tissue or probe different tissue volumes due to motion from bodily movement and activity, causing variations in the signal intensity and quality. When the device becomes decoupled from the tissue, an air gap can be created. This air gap can create a difference in the index of refraction which alters the signal intensity at the photodiode and results in signal loss. Currently, to minimize these noises and provide more light to the tissue, the physical form factor or embodiment is fastened tightly to create a tight coupling of the optical light source and photodiode of the device to the tissue. Increased pressure from tightening can reduce blood flow and corresponding light intensity from blood absorption at the photodetector. Additionally, tight fastening can be uncomfortable for the user during long periods of sleep or physical activity. In an embodiment, the device can be utilized during sleep apnea investigations.

According to embodiments of the disclosure, an adhesive interface device can adhere a wearable device to tissue and maintain optical coupling of the wearable device and the tissue. This optical coupling can reliably transmit light from the wearable device to the tissue and return light from the tissue to a photodetector. The adhesive interface device can improve the ability of the wearable device to continuously measure physiological measurements such as heart rate, blood oxygenation and deoxygenation, and other fitness and health measurements during physical activity. The adhesive interface device can be configured for a variety of wearable devices used in continuous fitness tracking and patient monitoring medical device applications. In an embodiment, the wearable optical sensor device is a photoplethysmograph, a cuffless blood pressure monitor, a pulse oximeter, a heart rate monitor, or a device for use related to sleep apnea. In an embodiment, the wearable optical sensor device is a watch, fitness wristband, rings, eyeglasses, a smartphone, finger clamp, or ear buds.

The adhesive interface device can be composed of a pressure sensitive adhesive (PSA) material. This PSA material can be applied as an adhesive film, with one surface adhering to the wearable device and another surface adhering to the tissue. The material can be index matched to have a similar index of refraction as the underlying tissue.

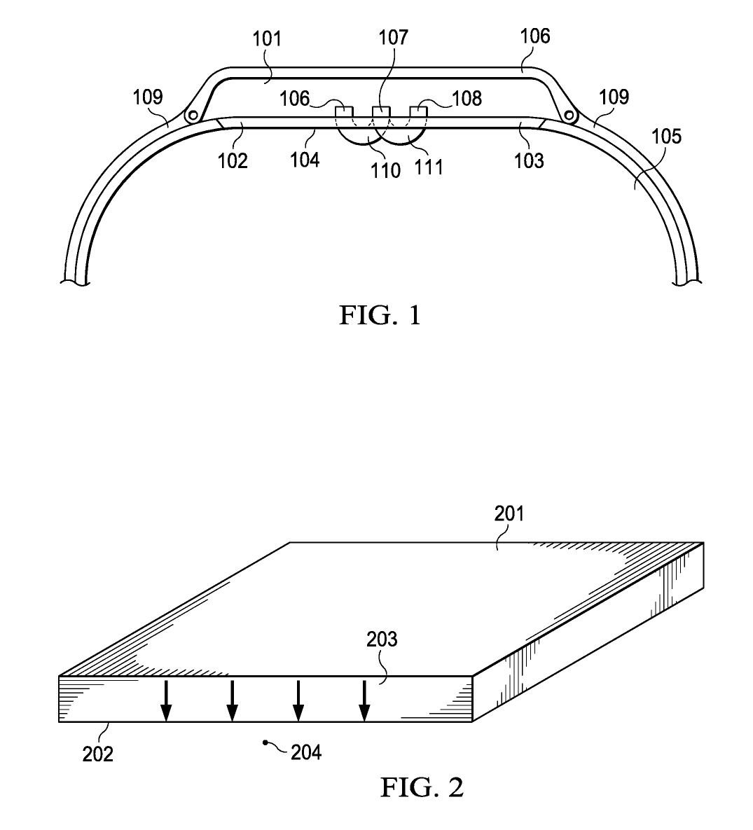

FIG. 1 is a cross-sectional view of an embodiment of the disclosed adhesive interface device 103 that illustrates movement of light through the adhesive interface device 103, according to embodiments of the disclosure. A smartwatch 101 is secured to tissue 105 with a wrist band 109 and coupled to the tissue 105 with the adhesive interface device 103. The adhesive interface material 103 adheres to the tissue 105 at a first surface 104 and adheres to the smartwatch 101 at a second surface 102. The adhesive interface device 103 acts as a light waveguide between the tissue 105 and smartwatch 101.

Light emitted from a first LED 106 and a second LED 108 transmits through the first surface 102, through the adhesive interface device 103, and through the second surface 104. Light from the first LED 106 can enter a first section of tissue 110 and light from the second LED 108 can enter a second section of tissue 111. Light from each LED 106 and 108 can be absorbed by blood in the respective sections of tissue 110 and 111 and transmitted back to a photodiode 107. As the blood volume in the sections of tissue 110 and 111 change, the light intensity that reaches the photodiode 107 changes. The difference in light intensity can be used to determine certain physiological measurements.

FIG. 2 is a three-dimensional cross-sectional view of a pressure sensitive adhesive interface device and light waveguide, according to embodiments of the disclosure. A second adhesive surface 201 and a first adhesive surface 202 are protected from exposure by liners before attachment to a surface of a wearable device and tissue 204. After attachment of the PSA interface device and during wearable device operation, light 203 enters the second surface 201 from an LED and is transmitted to the first surface 202. To minimize scattering of light away from the appropriate regions of the tissue 204, the PSA interface device can have a refractive index that matches that of the tissue 204.

The adhesive interface device can be comprised of materials selected for their adhesive and optical properties, in addition to other properties that can be favorable for proper wearable device operation. The device can be one material with both optical and adhesive properties, or can be an optical material with adhesive coated on the surface. An “adhesive” is any material which can usefully hold two or more objects together by intimate surface contact. A “pressure sensitive adhesive” (PSA) can designate a distinct category of adhesives which are tacky at room temperature and capable of firmly adhering to a variety of dissimilar surfaces upon mere contact without the need of more than tactile finger or hand pressure. A PSA can require no activation by water, solvent, or heat to exert a strong adhesive holding force toward such materials as plastic, silicon, and tissue. PSAs can have a sufficiently cohesive holding and elastic nature such that, despite their tackiness, they can be handled with fingers and removed from smooth surfaces.

A “patch adhesive” is a pressure sensitive adhesive that can contain one or more elastomers combined with resins or other components which impart tack, adhesion, cohesion, or other necessary properties. “Tack” is the condition of the adhesive when it is sticky, adhesive, and/or cohesive. PSA materials can include, but are not limited to, synthetic elastomers, hydrogels, polyethylene glycols, acrylics, rubbers, silicones, polyurethanes, polyesters, and polyethers.

The material can act as a light waveguide for transmitting light from one or more light sources to tissue and from tissue to a photodetector. The material can have a refractive index near that of human skin. Human skin typically has a real refractive index between 1.3 and 1.5. The refractive index can vary according to tissue properties, such as melanin concentration, skin thickness, and skin type. The refractive index can also vary depending on the wavelength of light contacting the skin. For example, skin usually has a lower refractive index for higher wavelengths of light. The material's refractive index can be configured and calibrated based on these and other tissue and device properties. The material can have a hole for transmission of light to the skin. The device can also include other materials or structures that can be used to guide, diffuse, filter, or scatter light transmitted between the wearable device and tissue. For example, the device can increase light transmission into the material by integrating other materials capable of scattering light.

The adhesive interface device can be configured to couple with a variety of wearable devices. The device can be cut from a sheet of the PSA material to fit onto the optical hardware of a wearable device. For example, an adhesive interface device can be a patch sized to fit onto the back of a smartwatch and covered with liner. A “liner” can be a waxy release paper behind an adhesive. In an embodiment, the liner is siliconized. The adhesive interface device can come in a variety of thicknesses configured for optical and physical characteristics, such as refractive index and tissue profile. For example, a thinner device profile can be desired for devices placed on flat or minimally changing tissue surfaces, such as a chest. In an embodiment, the adhesive is less than 1 mm thick. In an embodiment, the interface material is 1 mm or more thick. In an embodiment, the interface material is transparent. In an embodiment, the adhesive is 1 mm or more thick. In an embodiment, the adhesive is transparent. In an embodiment, the device is disposable. In an embodiment, the device can be removed from the wearable device and another device applied to the wearable device. In an embodiment, the second surface of the device can be applied to the wearable device with the liner present on the first surface until a time at which the liner is removed and the first surface of the device is applied to the tissue. In an embodiment, the device is removed after one use. In an embodiment, the device is removed after multiple uses.

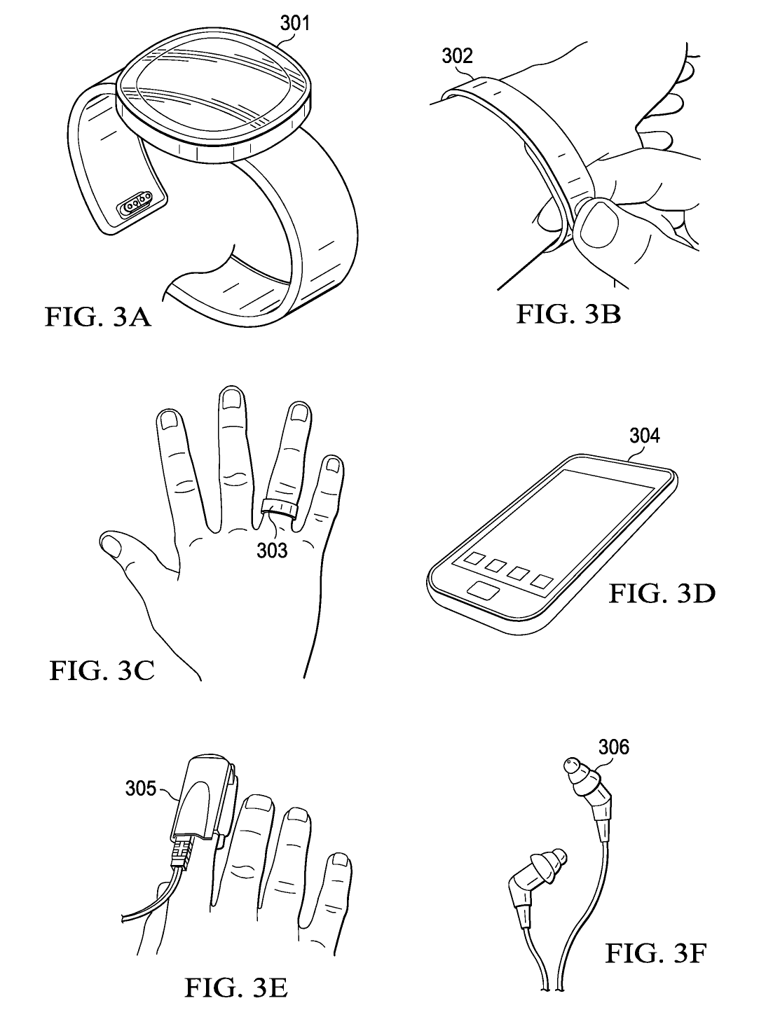

FIG. 3A-3F are illustrations of various wearable devices with which the adhesive interface device can be used, according to embodiments of the disclosure. Such wearable devices can include, but are not limited to, (A) a watch 301, (B) a fitness wristband 302, (C) a ring 303, (D) a smartphone 304, (E) a finger clamp 305, and (F) an ear buds 306.

Although the present composition and methods have been described in terms of specific embodiments, it is anticipated that alterations and modifications thereof will become apparent to those skilled in the art. Therefore, it is intended that the following claims be interpreted as covering all such alterations and modifications as fall within the true spirit and scope of the disclosure. All of the compositions and methods disclosed and claimed herein can be made and executed without undue experimentation in light of the present disclosure. While the compositions and methods of this disclosure have been described in terms of various embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are both chemically related can be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. An adhesive interface device, comprising:
an interface material having a first surface and a second surface;
wherein the first surface is configured to adhere to a tissue;
wherein the second surface is configured to adhere to a wearable optical sensor device; and
wherein the interface material has a refractive index similar to the tissue when illuminated.

2. The device of claim 1, wherein the first surface and the second surface of the interface material are adhesive.

3. The device of claim 2, wherein the interface material is less than 1 mm thick.

4. The device of claim 2, wherein the interface material is transparent.

5. The device of claim 2, wherein a liner covers the adhesive on the first surface and the second surface of the interface material.

6. The device of claim 5, wherein the liner is a waxy release paper.

7. The device of claim 1, wherein the tissue is illuminated by a LED.

8. The device of claim 1, wherein the refractive index of the interface material is between about 1.3 and 1.5.

9. The device of claim 8, wherein the refractive index of the interface material is about 1.3.

10. The device of claim 2, further comprising a first adhesive on the first surface and a second adhesive on the second surface.

11. The device of claim 2, wherein the interface material is a pressure sensitive adhesive.

12. The device of claim 11, wherein the pressure sensitive adhesive is at least one selected from the group consisting of synthetic elastomers, hydrogels, polyethylene glycols, acrylics, rubbers, silicones, polyurethanes, polyesters, or polyethers.

13. The device of claim 1, wherein the wearable optical sensor device is one of a smartwatch, a smartphone, a fitness band, eyeglasses, a heart rate monitor, an armband, a ring, or ear buds.

14. The device of claim 1, wherein the wearable optical sensor device is one of a photoplethysmograph, a cuffless blood pressure monitor, a pulse oximeter, a heart rate monitor, or a device for use related to sleep apnea.

15. The device of claim 1, wherein the device has a hole for light transmission for illuminating tissue with one or more wavelengths.

16. The device of claim 1, wherein absorption is measured using at least one photodetector.

17. The device of claim 1, wherein the interface material is configured to act as a waveguide or diffuser for illuminating tissue with one or more wavelengths.

18. The device of claim 1, wherein the material is configured to polarize or filter light for illuminating tissue with one or more wavelengths.

19. The device of claim 1, wherein the device is disposable.

20. The device of claim 1, further comprising a light scattering material to increase light transmission into the tissue.
